(19) United States
(12) Patent Application Publication
(10) Pub. No.: US 2006/0099166 A1
(43) Pub. Date: May 11, 2006

(54) Honey-based skin care preparation

(76) Inventor: Jan Vandeputte, Varsenare (BE)

Correspondence Address: LADAS & PARRY, 5670 WILSHIRE BOULEVARD, SUITE 2100, LOS ANGELES, CA, 90036-5679, US

(21) Appl. No.: 10/519,295

(22) PCT Filed: Jun. 19, 2003
(86) PCT No.: PCT/NL03/00451

§ 371 Date: Oct. 31, 2005

(30) Foreign Application Priority Data
Jun. 21, 2002 (BE) 2002/0405

(51) Int. Cl. A61K 8/81; A61K 8/73

(52) U.S. Cl. 424/70.13; 424/70.16

(57) ABSTRACT

The invention relates to a honey-based skin care preparation, wherein the honey is entrapped within an aqueous polymeric gel. The skin care preparation is suitable for healing wounds on the one hand and for cosmetic use on the other hand. Preferably, the skin care preparation comprises a humectant, such as glycerin, amongst other substances, in addition to honey and a polymeric gel. Furthermore, the polymeric gel may contain additional ingredients.

The invention relates to a honey-based skin care preparation.

Concerning skin care preparations, a wound dressing is known from U.S. Pat. No. 4,671,267 wherein a polymer matrix containing a water soluble humectant is used. Said document especially emphasizes the physiotherapeutic possibilities of the dressing, such as the “hot or cold” treatment. No mention is made therein of active or therapeutical ingredients. The term humectant is understood to mean a product that attracts moisture.

The drawback of such a skin care preparation is that the wound dressing effects a limited antibacterial activity. Such a wound dressing does not contain an active ingredient and it has no nutritional value for the wound.

From U.S. Pat. No. 5,980,875 there is known a method for the preparation of a honey-based formulation for topical application, comprising the forming of a base as a carrier and the mixing of active constituents of honey with the base, with the mixing of the active constituents further comprising the setting of a mixing temperature not exceeding 40° C. Although said US patent specification makes mention of the fact that a thickening agent may be added as a hydrophylic component, no further details with regard to the polymer gel are known therefrom.

From U.S. Pat. No. 6,174,535 there is known a hydrated honey gel polymeric composition, which composition comprises: polyglycerylmethacrylate and a liquid polysaccharide component selected from the group consisting of honey and corn syrup, wherein the weight ratio of the polyglycerylmethacrylate to the polysaccharide in the liquid polysaccharide component is from about 8:1 to about 1:2.

International patent application WO 02/30467 discloses the use of a honey-based composition as a therapeutically active ingredient, which composition furthermore comprises lanolin and/or a lanolin derivative, which is in particular suitable for treating wounds. The use of a polymeric gel is not known therefrom.

Concerning honey-containing skin care preparations, a composition comprising a therapeutically active compound with antiseptic, osmotic and other characteristics is known from U.S. Pat. No. 5,785,972, which composition is used in particular for the treatment of burns and open wounds experienced by animals and man, and in particular for the treatment of thermal burns on humans by use of spray composition. The composition comprises a combination of colloidal silver, helichrysum oil and raw honey emulsified with an emulsifier so as to form a solution, wherein water soluble lecithin is used as the emulsifier.

The drawback of such a skin care preparation is that a spray or a complex ointment will not remain present on a wound and will have to be covered with a secondary dressing at all times. As a result, the absorption of moisture does not take place directly from the wound.

A first aspect of the present invention is to provide a skin care preparation that does not exhibit the drawbacks as mentioned above.

A second aspect of the present invention is to provide a skin care preparation based on honey as a therapeutically active compound, which preparation is suitable for treating wounds, such as first and second degree burns, open wounds (chronic wounds), diabetic foot sores, bedsores (decubitus) and other skin surface wounds.

Yet another aspect of the present invention is to provide a skin care preparation having a special consistency or cohesion, wherein the honey as the active component can demonstrate the therapeutic effect thereof for a prolonged period of time, with a constant performance being ensured.

Another aspect of the present invention is to provide a skin care preparation which, when used on the human skin, will not stick to the skin tissue that forms the wound or to the surrounding skin tissue.

The present invention is characterized in that the honey is entrapped within an aqueous polymeric gel based on acrylic monomers or derivatives therefrom.

One or more of the aforesaid aspects are accomplished by using such a special polymeric gel. It has in particular been found that the present polymeric gel exhibits a very good moisture absorption capacity and a very stable cohesion without any dehydration or cracking of the polymeric gel, which might lead to additional wounds and irritation of the patient's skin.

Preferably, the skin care preparation according to the invention is suitable for healing wounds on the one hand and for cosmetic use on the other hand.

In one preferred embodiment of a skin care preparation according to the invention, the skin care preparation comprises

from 5 to 60 wt. % honey, and

from 30 to 40 wt. % polymeric gel

including 10-30 wt. % polymer, and

10-30 wt. % water,

based on the total amount of ingredients.

In a specific preferred embodiment of the skin care preparation according to the invention, the skin care preparation comprises a humectant in addition to honey and a polymeric gel.

The advantage of this is that it enhances the moisture absorption characteristics of the polymeric gel with honey entrapped therein.

The skin care preparation according to the invention preferably comprises

from 5 to 60 wt. % humectant,

from 5 to 60 wt. % honey, and

from 30 to 40 wt. % polymeric gel

including 10-30 wt. % polymer, and

10-30 wt. % water,

based on the total amount of ingredients.

In one preferred embodiment of a skin care preparation according to the invention, said humectant is glycerin.

The advantage of this is that besides the honey as the therapeutically active compound, glycerin has a synergetic effect as regards the antibacterial activity of the skin care preparation.

In order to obtain an optimum functioning of the present skin care preparation, the amount of honey preferably ranges in particular from 10 to 35%, based on the total amount of ingredients. Such an amount of honey, in combination with the special polymeric gel, provides a skin care preparation which is pleasant and soft to the touch, and which moreover exhibits a very quick absorption of moisture from the wound, which polymer does not disintegrate, not even after absorption of moisture.

In a very suitable skin care preparation, one or more monomers comprising 2-acrylamido-2-methylpropane sulphonic acid, acrylic acid, acrylic acid (3-sulphopropyl)ester, a substituted derivative thereof or a salt thereof are preferably used as acrylic monomers.

In such an embodiment, one or more monomers comprising at least one of acrylamide or a mono- or di-N-alkylacrylamide or an analog compound thereof containing an alkyl or a substituted alkyl group combined with a carbon-carbon double binding via an amido- or alkylamido function are used as acrylic monomers.

Preferred analog compounds are diacetone acrylamide, a vinyllactam, an N-alkylated acrylamide, an N,N-dialkylated acrylamide, N-vinylpyrrolidone or acryloylmorpholin.

The preparation of such a polymer is known to the skilled men in the art and need not be explained in more detail therein, therefore. It stands to reason that the preparation of such a polymer comprises the addition of a photo initiator, which is capable of generating free radicals, a cross-linking agent and water besides the use of the active monomer components, followed by the treatment of the mixture in such a manner, for example by irradiating it with light of sufficient intensity and the desired wavelength, that polymerisation and crosslinking of the mixture will take place. If desired, one or more other substances can be added to the mixture to be polymerised, for example softeners, colorants, stabilisers, surfactants and preservatives.

The polymeric gel of the skin preparation according to the invention preferably comprises 50 wt. % acrylamide and 50 wt. % water, based on the total amount of ingredients in the polymeric gel.

In one preferred skin care preparation according to the invention, said skin care preparation comprises one or more additional ingredients selected from the group of antioxidants, transretinoic acid and/or derivatives and precursors thereof, polyunsaturated fatty acids, n-hexacosanol, bis(maltolato)oxovanadium(IV), aloe vera or thickeners.

In a specific preferred skin care preparation according to the invention, the amount of additional components constitutes up to 40 wt. % of the total amount of ingredients.

In one advantageous embodiment of a skin care preparation according to the invention, the honey has a peroxide number of more than 5 μg/g honey/hour, measured at a temperature of 21° C.

In a very advantageous embodiment of a skin care preparation according to the invention, the honey has an LPS content of 14-130 EU/ml.

If the skin care preparation according to the invention is used for healing wounds, the honey is preferably subjected to a sterilisation treatment.

Preferably, said sterilisation treatment is carried out by means of gamma rays.

In one preferred embodiment of the skin care preparation according to the invention, the skin care preparation is in the form of a supple gel plate.

A skin care preparation according to the invention consists of a polymeric gel with honey being entrapped therein. This skin care preparation is quite suitable for healing acute wounds as well as chronic wounds, but it may also be used for cosmetic purposes.

The polymeric gel preferably consists of acrylic monomers, more specifically acrylamide and water in a proportion of 50/50 wt. %, based on the total amount of ingredients in the polymeric gel. The thickness of the polymeric gel may vary from 0.3 cm to 1.5 cm, in specific embodiments even from 0.2 to 2 mm.

Honey has a number of specific characteristics, such as being:

hyperosmolar;

trophic (=nutritional);

antibacterial;

debriding (in the case of scabbing of a wound, the scab will come loose and/or dissolve).

If honey and a polymeric gel are the only ingredients of the skin care preparation, the skin care preparation preferably comprises:

from 5 to 60 wt. % honey, and

from 30 to 95 wt. % polymeric gel

including 10-30 wt. % polymer, and

10-30 wt. % water,

based on the total amount of ingredients.

In addition to honey, the polymeric gel preferably comprises a humectant. A humectant is a substance that attracts moisture. The following products may be used as humectants: glycerin, polyethyleneglycol, dimethylsulphoxide and dimethylformamide, etc. A humectant is a substance which has a strong softening effect, which, on subjective evaluation, makes skin which is dry or rough due to an inadequate natural moisture retention, feel softer and better.

Preferably, glycerin is used. Glycerin thus contributes towards maintaining or restoring the required moisture level in the skin. Glycerin has a synergetic effect with honey as regards the antibacterial activity. Glycerin furthermore ensures that the suppleness of the polymeric gel is not lost.

In that case, the skin care preparation preferably comprises:

from 5 to 60 wt. % humectant,

from 5 to 60 wt. % honey, and

from 30 to 40 wt. % polymeric gel

including 10-30 wt. % polymer, and

10-30 wt. % water,

based on the total amount of ingredients.

The polymeric gel in combination with the honey and the glycerin provides an exchange system that works on the basis of gradient differences at a semi-permeable membrane. Honey and glycerin are more specifically hyperosmolar substances. The exchange system works as follows: moisture from the skin surface to be treated migrates towards the polymeric gel, whilst the honey and the glycerin migrates towards the skin surface to be treated. The antibacterial agent, in particular honey, is thus gradually released, whilst in addition excess wound moisture is absorbed in the polymeric gel.

In the cosmetic sphere, the skin preparation according to the invention acts on the epidermis of the skin. The glycerin effects a normalisation of the moisture level in the epidermis, whilst the honey removes free oxygen radicals via H₂O₂.

The skin care preparation preferably comprises one or more additional components selected from the group of antioxidants, transretinoic acid and/or derivatives and precursors therefrom, polyunsaturated fatty acids, n-hexacosanol, bis(maltolato)oxovanadium(IV), aloe vera or thickeners. Examples of thickeners are carboxymethyl cellulose or polyethylene glycol.

Especially preferred is vitamin A or a derivative thereof, for example a vitamin A-containing composition such as cod-liver oil. Vitamin A can be considered to be a precursor of vitamin A acid and has a positive effect on the healing process of wounds. The addition thereof is in particular desirable in the case of a retarded healing process of the patient, for example caused by the administration of corticosteroids. It is assumed that vitamin A or a derivative thereof stimulates the formation of type I and type III collagen and incites the epidermis cells to divide in an orderly manner.

Preferably, the skin care preparation comprises up to 40 wt. % additional ingredients, based on the total amount of ingredients.

The honey that is used in the skin care preparation according to the invention must have one or more special characteristics.

The honey must preferably have a peroxide number of more than 5 μg/g honey/hour, measured at a temperature of 21° C. If honey having a peroxide count outside the aforesaid range is used, the antibacterial activity of the honey will be insufficient.

Furthermore, the honey preferably has an LPS content (content of lipopolysaccharides) of 14-130 EU/ml (European units/ml).

Since the skin care preparation may be directly applied to the wound, it must furthermore be sterile. This can be effected by sterilising the entire amount of polymeric gel by means of gamma rays, for example gamma rays obtained from cobalt 60. Experiments have shown that the honey retains its antibacterial activity after being subjected to such radiation.

The honey must be free from heavy metals, pesticides and herbicides. These requirements are made because the skin care preparation is directly applied to the wound.

The following procedure is followed for preparing the skin care preparation:

the honey, the humectant and any additional ingredients are added to the liquid polymer;

the aggregate is mixed, preferably by means of a mixer;

a catalyst that initiates the chain reaction is added;

after activation, for example by irradiation with UV light having the desired wavelength or by heating, depending on the reaction system, the chain reaction is started within a few seconds to a few minutes, resulting in the forming of the polymeric gel.

The present invention will now be explained in more detail by means of a number of examples. It will be understood that no aspect of the following description is to be interpreted as limiting the scope of the invention as defined in the appending claims.

### EXAMPLE 1

### COMPARATIVE EXAMPLE

A person suffering from a severe case of decubitus was treated with pure honey, which was applied to the wound site. Although the healing process of the bedsores showed a slight improvement in comparison with the situation in which no honey was applied, the patient experienced undesirable pain. Part of the honey is quickly lost outside the wound area. Extra moisture being extracted from the wound runs off and must be absorbed by a secondary dressing.

Example of the Use of a Skin Care Preparation According to the Invention:

A composition consisting of 20 wt. % glycerin and 45 wt. % honey and 17.5% polyacryl matrix was used on a group of patients suffering from decubitus. The bedsores healed remarkably quickly, whilst in addition the patients did not experience the pain that was experienced in Comparative example 1. The dressing could remain in place longer because the release of the honey to the wound takes place gradually.

### EXAMPLE 2

Example of the Use of a Skin Care Preparation According to the Invention:

The composition of Example 1 was used, with this difference that additionally 10 wt. % cod-liver oil was incorporated therein. Patients treated with this composition experienced a very quick healing of their open wounds, whilst in addition their skin fully regained its smooth surface structure.

### COMPARATIVE EXAMPLE 2

A composition consisting of 17.5 wt. % acrylamide matrix and 65 wt. % glycerin was used with patients suffering from deep grazes/burns. Although this composition exhibited some therapeutic activity, the time it took for the wounds to heal was considerably longer than in the situation in which a composition that contained honey besides glycerin was used.

### EXAMPLE 3

Example of the Use of a Skin Care Preparation According to the Invention:

A composition consisting of 30 wt. % glycerin and 35 wt. % honey was used on a group of patients suffering from deep grazes/burns. The composition was experienced as pleasant by the patients and the wounds healed within two weeks.

1. A honey-based skin care preparation, characterized in that the honey is entrapped within an aqueous polymeric gel based on acrylic monomers or derivatives therefrom.

2. A skin care preparation according to claim 1, characterized in that the skin care preparation is suitable for healing wounds.

3. A skin care preparation according to claim 1, characterized in that the skin care preparation is suitable for cosmetic use.

4. A skin care preparation according to claim 1, characterized in that the skin care preparation comprises
from 5 to 60 wt. % honey, and
from 30 to 95 wt. % polymeric gel
including 10-30 wt. % polymer, and
10-30 wt. % water,
based on the total amount of ingredients.

5. A skin care preparation according to claim 1, characterized in that the skin care preparation comprises a humectant in addition to honey and a polymeric gel.

6. A skin care preparation according to claim 1, characterized in that the skin care preparation comprises
from 5 to 60 wt. % humectant,
from 5 to 60 wt. % honey, and
from 30 to 40 wt. % polymeric gel
including 10-30 wt. % polymer, and
10-30 wt. % water,
based on the total amount of ingredients.

7. A skin care preparation according to claim 5, characterized in that said humectant is glycerin.

8. A skin care preparation according to claim 1, characterized in that the amount of honey ranges from 10 to 35%, based on the total amount of ingredients.

9. A skin care preparation according to claim 1, characterized in that one or more monomers comprising 2-acrylamido-2-methylpropane sulphonic acid, acrylic acid, acrylic acid (3-sulphopropyl)ester, a substituted derivative thereof or a salt thereof are used as acrylic monomers.

10. A skin care preparation according to claim 1, characterized in that one or more monomers comprising at least one of acrylamide or a mono- or di-N-alkylacrylamide or an analog compound thereof containing an alkyl or a substituted alkyl group combined with a carbon-carbon double binding via an amido- or alkylamido function are used as acrylic monomers.

11. A skin care preparation according to claim 10, characterized in that the analog compound is diacetone acrylamide, a vinyllactam, an N-alkylated acrylamide, an N,N-dialkylated acrylamide, N-vinylpyrrolidone or acryloylmorpholin.

12. A skin care preparation according to claim 1, characterized in that the polymeric gel comprises 50 wt. % acrylamide and 50 wt. % water, based on the total amount of ingredients in the polymeric gel.

13. A skin care preparation according to claim 1, characterized in that said skin care preparation comprises one or more additional ingredients selected from the group of antioxidants, transretinoic acid and/or derivatives and precursors thereof, polyunsaturated fatty acids, n-hexacosanol, bis(maltolato)oxo-vanadium(IV), aloe vera or thickeners.

14. A skin care preparation according to claim 13, characterized in that the amount of additional components forms up to 40 wt. % of the total amount of ingredients.

15. A skin care preparation according to claim 1, characterized in that the honey has a peroxide number of more than 5 μg/g honey/hour, measured at a temperature of 21° C.

16. A skin care preparation according to claim 1, characterized in that the honey has an LPS content of 14-130 EU/ml.

17. A skin care preparation according to claim 1, characterized in that the honey is subjected to a sterilisation treatment when used for healing wounds.

18. A skin care preparation according to claim 17, characterized in that said sterilisation treatment is carried out by means of gamma rays.

19. A skin care preparation according to claim 1, characterized in that said skin care preparation takes the form of a supple gel plate having a thickness of about 0.2-2 mm.
